(19) United States
(12) Patent Application Publication
(10) Pub. No.: US 2015/0367581 A1
(43) Pub. Date: Dec. 24, 2015

(54) METHODS AND DEVICES FOR BREAST IMPLANT SURGERY AND SELECTION

(71) Applicant: Michael Tantillo, Weston, MA (US)

(72) Inventor: Michael Tantillo, Weston, MA (US)

(21) Appl. No.: 14/745,471

(22) Filed: Jun. 22, 2015

Related U.S. Application Data
(60) Provisional application No. 62/015,424, filed on Jun. 21, 2014.
(60) Provisional application No. 62/015,409, filed on Jun. 21, 2014.

(51) Int. Cl. B29C 67/00; G05B 19/4099; A61F 2/12

(52) U.S. Cl. CPC B29C 67/0088; A61F 2/12; G05B 19/4099; A61F 2240/002; B33Y 30/00

(57) ABSTRACT

The present disclosure provides several processes, algorithms, and devices for breast implant surgery. An algorithm of the present disclosure can receive input from a user, surgeon, patient, and determine an appropriate or desired implant size or shape. The algorithm can prompt the user for adjustment and/or prioritization of certain parameters, and adjust the implant determination accordingly. Two-dimensional (2D) sizers of the present disclosure can be used during implant surgery to assist in determining an appropriate amount of pocket dissection. The 2D sizers can be inserted into a partially dissected cavity to determine whether the cavity is the correct size, and the doctor can then perform more dissection if needed. The processes and devices of the present disclosure can also be used with other imaging devices and a printer to provide implants on site.

### CROSS-REFERENCE TO RELATED APPLICATIONS

The present applications claims the benefit of U.S. Provisional Patent Application Ser. Nos. 62/015,424, filed on Jun. 21, 2014, and 62/015,409, filed on Jun. 21, 2014, each of which is incorporated by reference in their entirety.

### BACKGROUND OF THE DISCLOSURE

1. Field of the Disclosure

The present disclosure relates to methods and devices for breast implant pocket design and selection, and the accompanying surgery. More particularly, the present disclosure related to customized two-dimensional (2D) sizers for breast augmentation or reconstruction with prostheses, and an algorithm for selecting an implant size.

2. Description of the Related Art

In plastic surgery, especially when implanting textured round and shaped breast implants, it is necessary for exact measurements. Guides or footprints are necessary to create appropriate sized pockets for the implant both for aesthetic reasons as well as patient safety. In a breast implant surgery, the surgeon will dissect a pocket in the patient's breast, for placement of the implant. Inaccurate pocket dissection (both under dissection and over dissection) can increase the risk of complications, particularly when textured devices are used. In current practice three-dimensional (3D) guides are often used.

These 3D disposable guide implants are very expensive, adding to the cost of surgery. A full 3D disposable implant also does not provide sufficient flexibility or space for the surgeon to work around. Alternatively, placing the permanent implant without complete precise pocket dissection can be potentially damaging to the implant. Removal and replacement of the permanent implant can potentially weaken the shell from shear forces. Using electrocautery to complete the dissection with the implant in situ can potentially cause thermal damage to the implant shell. Completing the pocket by blunt dissection with the implant in place can increase the risk of bleeding which in turn increases the risk of capsular contracture.

In addition, in cosmetic and reconstructive plastic surgery, it is necessary to understand fully the patient's goals, and accurately decide upon the size and shape of implant that will meet those goals and is appropriate and safe to implant. Despite years of training, this practice remains a difficult art, which may lead to errors of judgment. There are currently no dynamic and interactive breast implant planning aids to assist in the process of selecting the proper, optimal implant.

The present disclosure addresses these deficiencies.

### SUMMARY OF THE DISCLOSURE

The present disclosure provides an application and algorithm that allows a surgeon to determine the appropriate size, shape, and volume for a breast implant based on several factors relating to the patient undergoing the procedure. These factors are discussed in greater detail below. The application and algorithm of the present disclosure will make an objective determination of the appropriate implant based on these factors, and display several options for the physician or surgeon to discuss with the patient. The application can be run on any number of computing and display devices, and accessed from a variety of locations.

The present disclosure also provides two-dimensional (2D) sizers for use on breast surgeries. The 2D implants can be implanted into the patient's breast pocket while the pocket is only partially dissected. The 2D implants have a minimal thickness, so they are more flexible and less costly than currently used 3D temporary pocket implants.

The present disclosure also provides an application and algorithm that allows a surgeon to determine the appropriate size, shape, and volume for a breast implant based on several factors relating to the patient undergoing the procedure. These factors are discussed in greater detail below. The application and algorithm of the present disclosure will make an objective determination of the appropriate implant based on these factors, and display several options for the physician or surgeon to discuss with the patient. The application can be run on any number of computing and display devices, and accessed from a variety of locations.

Thus, in one embodiment, the present disclosure provides a system for selecting a breast implant. The system comprises a server, an input/output device in communication with the server, the device comprising a user interface, and an algorithm. The algorithm is resident on the server, the device, or both of the server and the device. The algorithm prompts a user to input one or more parameters relating to the breast implant into the device through the user interface, and calculates at least one of a volume, diameter, projection, and shape of the breast implant based on the user input. The algorithm also provides an output to the user interface.

The present disclosure also provides a method of selecting characteristics of a breast implant, comprising the steps of interviewing a patient who will receive the implant, using the above-described system to calculate the characteristics of the breast implant, and consulting the patient with the characteristics.

The present disclosure also provides a method of performing a breast implant surgery, comprising the steps of performing a first dissection on a patient, to create a cavity to receive the implant, inserting a sizer into said cavity, to determine if any additional dissection is needed, performing any required additional dissection, if necessary, removing the sizer from the cavity, and inserting the implant into the cavity. The sizer can be flat, with a substantially uniform thickness. The method can also comprise the step of accessing a database of data relating to a plurality of breast implant footprint sizes, and printing the sizer based on the accessed data. The method can also comprise the step of acquiring image data relating to a plurality of breast implants, and printing the implant based on the accessed data.

### BRIEF DESCRIPTION OF THE DRAWINGS

FIG. 1 shows a flow chart illustrating a first process of the present disclosure, using an algorithm of the present disclosure.

FIG. 2 shows a schematic diagram of a system configuration using the process and algorithm of FIG. 1.

FIG. 3 shows a screenshot of a user interface for executing the process and algorithm of FIGS. 1 and 2.

FIG. 4 shows a flow chart illustrating a process for using the two dimensional (2D) sizers of the present disclosure.

FIG. 5 shows a sample of a 2D sizer according to the present disclosure.

FIG. 6 shows a side view of the sizer of FIG. 5.

FIG. 7 shows a flow chart for using the process of FIGS. 1 and 4 in conjunction with an apparatus for printing an implant.

### DETAILED DESCRIPTION OF THE DISCLOSURE

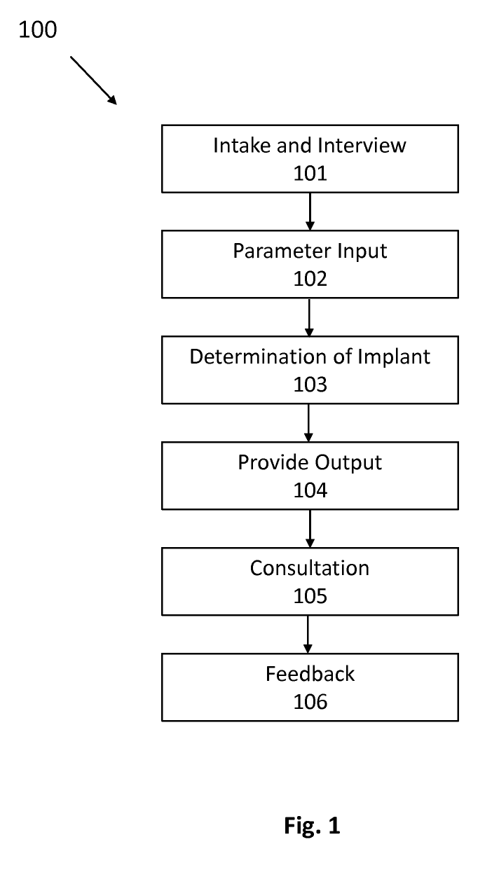

Referring to the drawings, and in particular FIGS. 1-3, a first process 100 using an algorithm 101 of the present disclosure is shown. System 1 can comprise algorithm 110, device 120, and server 130. Algorithm 101 can be resident on device 120, or in cloud or server 130. Alternatively, a first portion of algorithm 110 can reside on device 120, and a second portion can reside on server 130. As discussed in greater detail below, device 120 can be any computing device capable of running algorithm 110 and communicating with server 130 as needed. Algorithm 110 provides or runs through a user interface 112 on device 120. A user, such as a doctor, physician, or surgeon can input desired parameters (described in greater detail below) into algorithm 110 through interface 112. These parameters can relate to a desired or optimal size of a breast implant for a patient. Algorithm 110 will then determine one or more acceptable implant candidates based on these inputs.

Process 100, in conjunction with algorithm 110, saves significant time and money for the user. It can very precisely determine an implant shape and size, which can eliminate subjective errors or misjudgments. Additionally, process 100 and algorithm 110 allow the user to explain to patients how they came to their conclusion on implant size and shape, which will further aid in the decision making process for both the user and the patient. Use of algorithm 110 will also reduce the incidence of secondary surgery to change implant size and/or style.

As shown and described in FIG. 1, during a first step 101 of process 100, a surgeon or other user will conduct a full patient intake and determine the patient's aesthetic goals and their physical characteristics relevant to an optimal breast implant selection. The surgeon or user can also input parameters relating to the surgeon's preferences and expertise, in step 102. Accordingly, process 100 and algorithm 110 of the present disclosure can take into account many factors, such as, but not limited to, the patient's aesthetic goals, simultaneous breast lift surgery, the patient's skeletal and soft tissue characteristics, the manufacturer of the implant, the implant volume, the implant shape, the implant height to implant width relationship, the implant fill material, the implant shell type, and the implant projection.

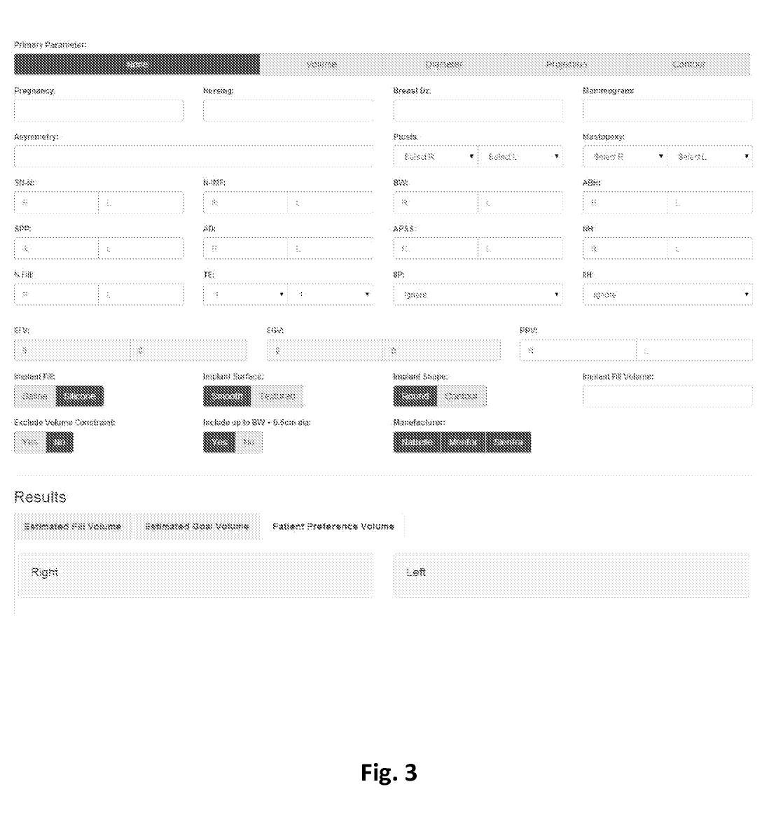

Algorithm 110 then determines one or more suitable implants based on the input parameters (step 103), and provides an output 114 on user interface 112 for the user and patient to review (step 104). As shown in FIG. 3, algorithm 110 also allows for prioritization of implant variables. For example, the user can prioritize the implant projection, the implant shape or the implant volume.

The output 114 displayed on interface 112 can show an estimated goal volume and shape of the eventual implant, which can then be compared to the patient's desired goal volume. This allows the surgeon or user to discuss the options of what these differences will mean both medically and aesthetically to the patient (step 105). In one embodiment, in a feedback step 106, algorithm 110 can allow the surgeon to choose or manipulate a primary parameter, which then changes the importance of all further parameters.

Furthermore, algorithm 110 can provide a ranking based on the priority of importance of one or two factors from the surgeon and/or patients' perspective, which then changes the ability of the user to select other criteria based on their primary goal. Breast implants usually have four basic characteristics: volume, diameter (width on the chest wall), projection (out from the chest wall), and shape (contour or round). Once the surgeon decides which of these characteristics is most important for the particular patient, then algorithm 110 will automatically restrict any implant choices that are incompatible with the primary parameter. For example, if the surgeon decides that the patient would be best served with a high projection implant and selects that as the primary parameter, then algorithm 110 will only offer high projection implants as choices. Algorithm 110 can perform this restriction either in steps 102 or 106. This novel approach will further aid the surgeon or user in selecting the correct implant based on criteria that are critical in meeting both medical safety and patient desired aesthetics.

Process 100 and algorithm 110 of the present disclosure can display output 114 in the form of one or more of the following three implant volumes. The Estimated Fill Volume (EFV) is based on the physical examination parameters entered into algorithm 110, and is thought to be the ideal implant volume to minimize long term soft tissue complications. Stated another way, the EFV is the volume beyond which the implant is placing undue stress on the breast tissue, and increasing the risk of revision being required in the future. The Estimated Goal Volume (EGV) is based on the physical examination parameters and also takes into account the patient's aesthetic goals. The EGV is also the volume that algorithm 110 calculates, based both on the subjective aesthetic goals of the patient and the objective physical examination measurements, and will best satisfy the patients aesthetic wishes or goal. The Patient Preference Volume (PPV) is the volume that the patient ultimately prefers based on their own decision. The EGV allows the surgeon to tell the patient that based on what they are telling the surgeon, this is the volume needed to achieve her goals. If the EGV is greater than the EFV, the surgeon can clarify for the patient that this goal will place stress on the tissues. When the PPV is different than the EGV, it allows the surgeon to review with the patient her aesthetic goals to clarify the patients goals (and revise the EGV), or recognize that the patient is accepting some sort of compromise.

Algorithm 110 delivers the best implant choices for each of these volumes prioritized by any constraints imposed by the user. Algorithm allows the user to change prioritization dynamically, as in feedback step 106. For example, the user can choose to prioritize a contour implant over a round implant, choose to prioritize moderate profile implants, or other features relating to the implant. The user can enter any number of constraints provided they are not mutually exclusive. For example, all contour shaped implants have a textured implant shell, so the user may not be able to constrain algorithm 110 to choose contour implants with a smooth shell; nor can the use constrain algorithm 110 to choose round implants and also implants that are taller than they are wide. The user can, however, constrain algorithm 110 to choose implants are taller than they are wide (and therefore contour implants and therefore textured implants) and implants that are of moderate projection. The dynamic nature of algorithm 110 allows the surgeon to counsel the patient about implant choices that may increase long term complications and then constrain algorithm 110 against those choices. Algorithm 110 will provide an important tool for surgeons to help patients make safer implant choices, including those choices that may otherwise lead to secondary surgery for implant size and/or style change.

FIG. 3 shows an example screen capture of user interface 112. As can be seen, there can be several pull-down or input slots for the user to input desired parameters relating to the implant and surgical procedure. The output 114 as described above will be shown as the parameters are entered. FIG. 3 shows the inputs for patient aesthetic goals, simultaneous breast lift surgery and detailed fixed tissue and dynamic tissue examination.

Device 120 can be selected from any number of computing and display devices, such as tablets, laptops, desktop computers, or smart phones. Process 100 and algorithm 110 can be run through web- or browser-based applications, or on an application accessed through an app store (e.g., Google Play, or the iTunes App Store) and run on a device 120. Process 100 and algorithm 110 of the present disclosure can also be a downloadable desktop client program that resides locally on a user's machine.

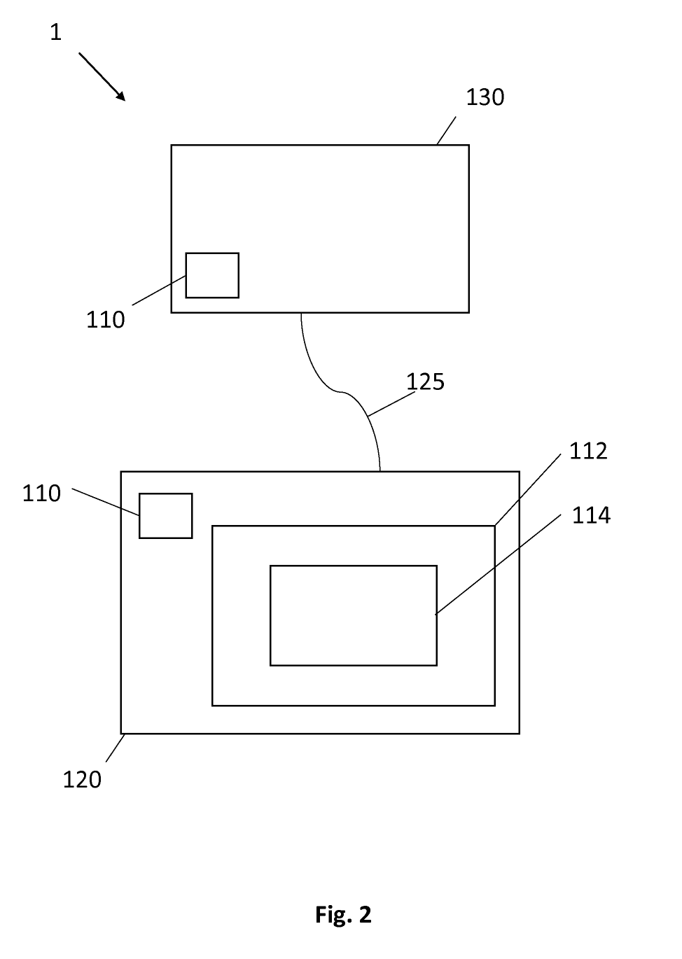

As shown in FIG. 2, in system 1, device 120 can communicate with server 130 via link 125. As described algorithm 110 may reside on device 120, server 130, or both, depending on the processing power of device 120. For example, if device 120 is a desktop computer, it may be able to run algorithm 110 locally, and may only need to communicate with server 130 occasionally (e.g., for updates to algorithm 110). Alternatively, if device 120 is a smart phone with limited processing power, it may be able to run a first portion of algorithm 110 locally, and communicate with sever 130, so that a second portion of algorithm 110 can perform larger calculations on server 130. Communications link 125 can be a cellular, wireless, or cable connection, or any other suitable type of connection.

The application also has the potential to be integrated directly into an electronic medical record system.

The present disclosure also provides a second process 200 creating a breast implant pocket design. Process 200 allows for breast implant footprints to be printed in a cost-effective manner and pass on much needed savings in the medical industry. In process 200, a surgeon can use a two-dimensional (2D) sizer 210 to create an appropriately sized pocket for breast surgery. The 2D sizer 210 is more flexible than their 3D counterparts. As previously discussed, this allows the surgeon to place the 2D sizer into the breast pocket, and size it accordingly before the dissection of the pocket is complete. Once the 2D sizer is in place, the surgeon can complete the dissection. This leads to more accurate creation of the pocket, and thus safer surgeries, with less risk of long term complications leading to additional surgeries.

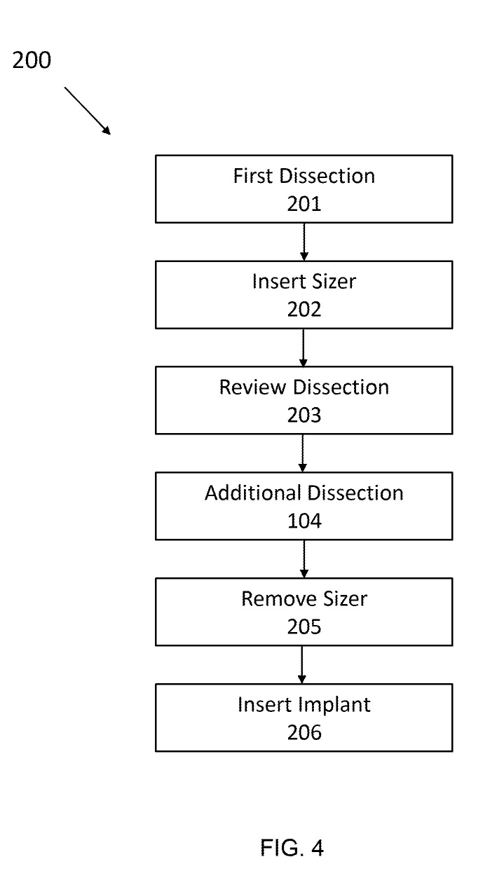

As shown in FIG. 4, during process 200, a surgeon or user can make a first dissection in the patient to form a cavity (step 201). The surgeon can then insert sizer 210 into the dissected cavity (step 202). The surgeon can then determine whether any additional dissection needs to be performed (step 203). If additional dissection is required, the surgeon can perform the second dissection (step 204). Steps 203 and 204 can then be repeated if necessary. Once the proper amount of cavity dissection is achieved, the user can remove sizer 210 (step 205), and place the implant into the dissected cavity (step 206).

Process 200 thus addresses an unfulfilled need for cost-efficient sizers in a very common surgery. Sizers 210 are thin and can be shipped to the surgeon in multiples of pre-sterilized packages. Given the cost-efficient manner that these will be produced the surgeon can have multiple different sizes in the operating room to aid in surgery without being overly concerned with the wasted overhead of using multiple expensive pocket sizers currently available today. This will provide added benefit to the patient, as sometimes a surgeon's decision on pocket size changes during surgery given multiple unknown factors.

The 2D sizers 210 of the present disclosure also allow the surgeon to precisely dissect the pocket without worry of either over dissection or under dissection. Over-dissection of the implant pocket can lead to seroma in the setting of a textured (round or shaped) implant or implant rotation (shaped implants). Pocket under-dissection requires the surgeon to either remove and replace the permanent implant or complete the pocket dissection with the permanent implant in place. Either scenario is potentially damaging to the implant and risky to the patient. Removal and replacement of the implant can potentially weaken the implant shell by shear forces. Electrocautery dissection with the implant in the pocket can potentially weaken the implant shell by thermal injury. Blunt pocket dissection with the implant in the pocket can increase bleeding which increases the risk of capsular contracture. The 2D sizer of the present disclosure can eliminate or mitigate all of these deficiencies.

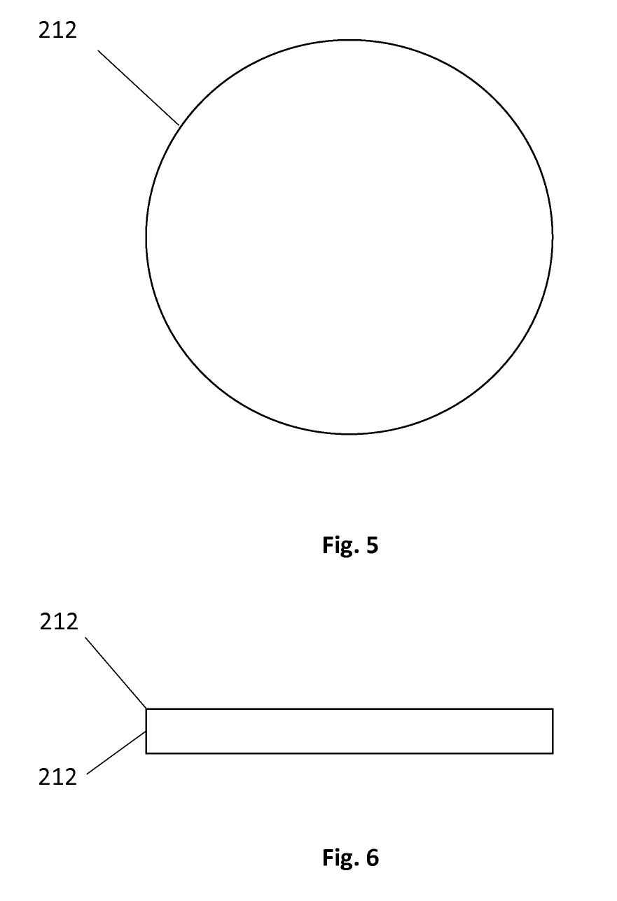

By “two-dimensional”, the present disclosure does not mean that the sizers 210 of the present disclosure have zero thickness. Clearly, the sizers 210 will have some minimal thickness 212 that provides structural integrity while still retaining the flexibility that is key. Sizers 210 of the present disclosure are substantially or completely flat. Sizers 210 can also have a uniform thickness 212. Thickness 212 can be from three to seven millimeters, or any sub ranges therebetween. Is 3-5 mm still a suitable range? Wider thickness possible? Sizer 210 can also have a number of shapes, including but not limited to round, elliptical, oval, oblong, tear-shaped, or any other shape that will facilitate insertion of sizer 210 into the dissected cavity as described above. An example of a 2D sizer according to the present disclosure is shown in FIGS. 5 and 6.

The materials suitable for sizer 210 may vary but would be of a biocompatible nature. The materials can include polylactic acid and acrylonitrile butadiene styrene, or silicone, which are approved by the United States Food and Drug Administration for implantable devices. Other materials contemplated may include rubber, light-cured polymers, metals, and implantable antibiotic-impregnated solids.

Process 100 and process 200 of the present disclosure may be used in conjunction with one another. For example, a user or surgeon could use process 100 and algorithm 110 to determine an appropriate implant for surgery. The surgeon could then perform process 200 with sizer 210.

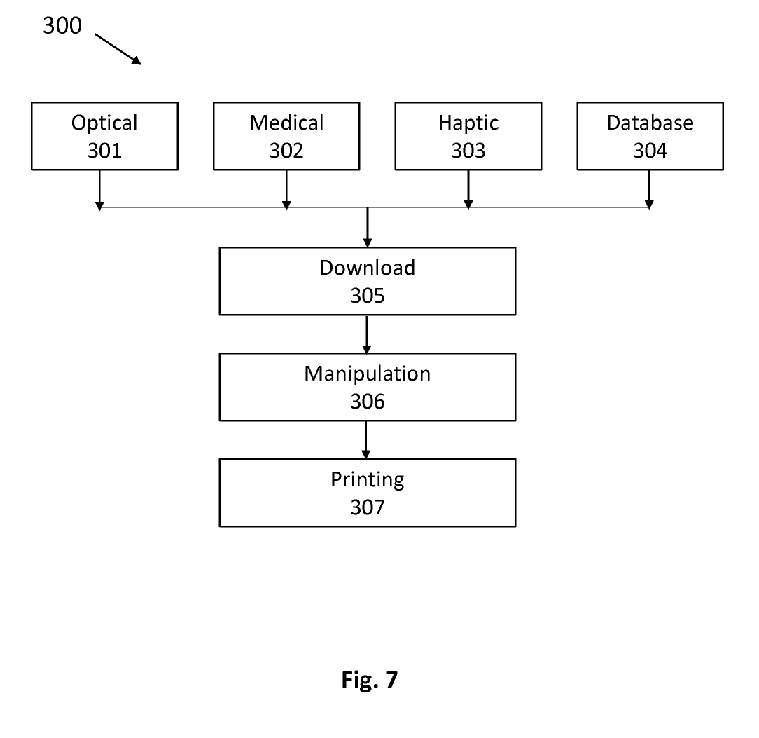

Either or both of process 100 and process 200 could be used in conjunction with a third process 300, shown and described in FIG. 7. In process 300, a digital image can be acquired via one of several methods. These include optical imaging, such as with a camera or three-dimensional scanning device (step 301), through medical imaging devices such as CT scanning or MRI devices (step 302), with haptic mapping (step 303), or by acquiring an image of an implant from a database of stored implant models (step 304). The digital image data acquired in any of steps 301-304 could then be downloaded to a storage or display device (step 305). Process 300 then allows the user, physician, surgeon, and/or patient to manipulate the image shown on the device of step 305 (step 306). Once a suitable final image is decided on, the final image could be sent to a three-dimensional printer to produce the implant (step 307).

Process 300 thus adds beneficial options and customizations to use in conjunction with either or both of processes 100 and 200. For example, if process 300 were used in conjunction with process 100, algorithm 110 can receive and process any of the image data acquires in process steps 301-303, or use server 130 to acquire an image from a remote database as in step 304. If process 300 were used in conjunction with process 200, sizer 210 could be printed on site, as in step 307. Sizers 210 could also be printed on site, at the time of or immediately before the procedure is to be performed. Sizers 210 of the present disclosure can also be used in conjunction with more traditional three-dimensional (3D) disposable implants. One could use sizer 210 of the present disclosure for pocket dissection, and then a 3D sizer for volume assessment.

The devices and methods of the present disclosure are discussed in the context of two- or three-dimensional printing, also known as “additive manufacturing”. However, any of processes 100-300 may employ the process of “subtractive” manufacturing as well. In this embodiment, the image acquisition device would send an image of a desired prosthesis to the computer, as described above. The final image, with or without modification, is sent to a fabricator. The fabricator uses subtractive methods to produce the prosthesis, where the prosthesis can be hewn from a solid piece of implantable material.

In addition to being suitable for implanting prostheses in patients, the devices and methods of the present disclosure can provide surgical planning models for the doctor and patient. The doctor can hold a model of a breast implant, for example, and develop a plan of appropriate sizing for the patient and these models can be printed quickly and inexpensively. This disclosure addresses the need to be able to print footprints for breast augmentation that can be easily sterilized and would greatly cut the cost of breast surgery.

Although the present disclosure is described in terms of breast surgery and augmentation, the methods, interfaces, and algorithms of the present disclosure could be adapted to other surgical implants or procedures. For example, the algorithm could be adapted to two stage tissue expander/implant breast reconstruction after mastectomy.

While the present disclosure has been described with reference to one or more particular embodiments, it will be understood by those skilled in the art that various changes may be made and equivalents may be substituted for elements thereof without departing from the scope thereof. In addition, many modifications may be made to adapt a particular situation or material to the teachings of the disclosure without departing from the scope thereof. Therefore, it is intended that the disclosure not be limited to the particular embodiment(s) disclosed as the best mode contemplated for carrying out this disclosure.

What is claimed is:

1. A system for selecting a breast implant, the system comprising:
a server;
an input/output device in communication with said server, said device comprising a user interface; and
an algorithm, wherein said algorithm is resident on said server, said device, or both of said server and said device,
wherein said algorithm prompts a user to input one or more parameters relating to the breast implant into said device through said user interface, and calculates at least one of a volume, diameter, projection, and shape of the breast implant based on said user input.

2. The system of claim 1, wherein said algorithm provides an output to said user interface.

3. The system of claim 2, wherein said algorithm allows the user to customize said output.

4. The system of claim 1, wherein said algorithm allows the user to prioritize one or more of said parameters before making said calculation.

5. The system of claim 1, wherein said algorithm calculates an estimated fill volume representing a maximum ideal volume of the implant based on said parameters, and an estimated goal volume based on a user preferences for said parameters.

6. The system of claim 1, wherein said device is a mobile smart phone.

7. The system of claim 1, wherein said one or more parameters are selected from the group consisting of a patient's aesthetic goals, whether the patient is having simultaneous breast lift surgery, a patient's skeletal and soft tissue characteristics, a manufacturer of the implant, an implant volume, an implant shape, an implant height to implant width ratio, an implant fill material, an implant shell type, an implant projection, and any combinations thereof.

8. The system of claim 1, further comprising a printer in communication with said server and said device, wherein said algorithm communicates said calculated shape and/or volume to said printer, for printing of the implant.

9. A method of selecting characteristics of a breast implant, comprising the steps of;
interviewing a patient who will receive the implant;
using the system of claim 1 to calculate the characteristics of the breast implant; and
consulting the patient with said characteristics.

10. The method of claim 9, further comprising the step of providing feedback to the algorithm of claim 1 via said user interface.

11. The method of claim 9, further comprising the step of printing the implant based on said characteristics.

12. A method of performing a breast implant surgery, comprising the steps of:
performing a first dissection on a patient, to create a cavity to receive the implant;
inserting a sizer into said cavity, to determine if any additional dissection is needed;
performing any required additional dissection, if necessary;
removing said sizer from said cavity; and
inserting the implant into said cavity.

13. The method of claim 12, wherein said sizer is flat, with a substantially uniform thickness.

14. The method of claim 13, wherein said sizer has a shape selected from the group consisting of round, oval, elliptical, or tear-shaped.

15. The method of claim 13, wherein said thickness is between three and five millimeters.

16. The method of claim 12, wherein said sizer is made from a material selected from the group consisting of polylactic acid, acrylonitrile butadiene styrene, silicone, rubber, light-cured polymers, metals, and antibiotic-impregnated solids.

17. The method of claim 12, wherein said sizer is made from a material selected from the group consisting of polylactic acid, acrylonitrile butadiene styrene, and silicone.

18. The method of claim 12, further comprising the step of printing said sizer with a printer.

19. The method of claim 18, further comprising the step of accessing a database of data relating to a plurality of breast implant footprint sizes, and printing said sizer based on said accessed data.

20. The method of claim 12, further comprising the step of acquiring image data relating to a plurality of breast implants, and printing the implant based on said accessed data.
